(19) United States
(12) Patent Application Publication
(10) Pub. No.: US 2006/0035901 A1
(43) Pub. Date: Feb. 16, 2006

(54) Methotrexate derivatives useful for treating cancer and arthritis

(75) Inventors: Gunnar J. Hanson, Chapel Hill, NC (US); Thomas E. Barta, Carrboro, NC (US); James Marvin Veal, Apex, NC (US); Roy Wendell Ware JR., Raleigh, NC (US)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Serenex, Inc., Durham, NC (US)

(21) Appl. No.: 11/186,404

(22) Filed: Jul. 21, 2005

Related U.S. Application Data
(60) Provisional application No. 60/589,696, filed on Jul. 21, 2004.

(51) Int. Cl. C07D 475/02; A61K 31/525

(52) U.S. Cl. 514/251; 544/259; 544/260

(57) ABSTRACT

The present invention provides novel derivatives of MTX. The novel derivatives have increased selectivity, inhibit the activity of Her2, EGFR, or B-Raf to a greater degree than MTX, and display greater antiproliferative activity than MTX. Also provided are pharmaceutical compositions containing the novel derivatives, to methods of preparation of such derivatives, to methods of inhibiting the biological activity of polypeptides, methods of inhibiting cell proliferation, and the use of the novel compounds in therapy, particularly for the treatment of neoplastic, hyperproliferative, and immune disorders, including cancer, arthritis, and psoriasis.

### CROSS-REFERENCE TO RELATED APPLICATION

This application claims the benefit of U.S. Provisional Application No. 60/589,696, filed Jul. 21, 2004, which is incorporated herein in its entirety.

### FIELD OF THE INVENTION

The present invention relates to novel derivatives of methotrexate, to pharmaceutical compositions containing them, to methods of preparation of such derivatives, to methods of inhibiting the biological activity of polypeptides, methods of inhibiting cell proliferation, and to their use in therapy, particularly in the treatment of neoplastic, hyperproliferative, and immune disorders, including cancer, arthritis, and psoriasis.

### BACKGROUND OF THE INVENTION

Methotrexate (MTX) is a folate antagonist that inhibits the folate-dependent enzyme dihydrofolate reductase (DHFR), and has also been shown to directly inhibit the activity of thymidylate synthase (TS) and phosphoribosylglycinamide formyltransferase (GART) (Purcell and Ettinger (2003) Current Oncology Reports 5:114-125). The compound is known by the chemical nomenclature N-[4-[[(2,4-diamino-6-pteridinyl)methyl]methylamino]benzoyl]-L-glutamic acid and has the structure shown in formula (I).

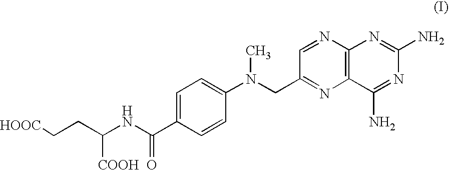

MTX has been used in the treatment of a number of types of cancer, including lymphoblastic leukemia, meningeal leukemia, lymphoma, choriocarcinoma, osteosarcoma, mycosis fungoides, Burkitt's and other non-Hodgkins' lymphomas, and carcinomas of the breast, hand, neck, ovary, and bladder. In addition to its use in cancer chemotherapy, MTX is a potent inhibitor of cell-mediated immune reactions and has been used as an immunosuppressive agent. MTX is currently among the most commonly used treatments for rheumatoid arthritis (Weinblatt et al. (1985) N. Eng. J. Med. 312:818-322; and Williams et al. (1985) Arthritis Rheum. 28:721-730), and is also used to treat other chronic inflammatory disorders. MTX is also effective in the prophylaxis of acute graft-versus-host disease either alone or in association with cyclosporin A and/or prednisone (Storb et al. (1986) N. Engl. J. Med. 314:729-35; Nash et al. (1992) Blood 80:1838-45; and Chao et al. (1993) N. Engl. J. Med. 329:1225-30) or FK506 (Nash et al. (1996) Blood 88:3634-3641), and is used in an adjunct therapy for persistent mild cardiac allograft rejection (Olsen et al. (1990) Transplantation 50:773-75). Other immune disorders in which MTX is used include dermatomyositis, rheumatoid arthritis (Hoffmeister (1983) Am. J. Med. 30:69-73), Wegener's granulomatosis, Crohn's disease (Feagan et al. (1995) N. Eng. J. Med. 332:292-7), and multiple sclerosis and associated disorders of the central nervous system (U.S. Patent Application No. 20030008875). MTX has also been used to treat the abnormally rapid proliferation of epidermal cells associated with psoriasis (McDonald (1981) Pharmacol. Ther. 14:1-24).

MTX is the folate antagonist that is most commonly used in the treatment of neoplastic, hyperproliferative, and immune disorders. However a number of adverse effects are associated with MTX treatment, particularly when higher doses of MTX are used. These adverse effects include myelosuppression, alopecia, dermatitis, interstitial pneumonitis, nephrotoxicity, defective oogenesis or spermatogenesis, abortion, and teratogenesis. Hepatic dysfunction, usually reversible but sometimes leading to cirrhosis, also occurs in some cases. Intrathecal administration of MTX can cause meningismus and an inflammatory response in the cerebrospinal fluid. Seizures, coma, and death result in rare instances. Similarly, adverse effects observed with the folate antagonists trimetrexate, edatrexate, raltitrexed, premetrexed, GW1843, OSI-7904L, nolatrexed, ZD9331, lomotrexol, and LY309887 include myelosuppression, rash, mucositis, fever, diarrhea, nausea, vomiting, transaminitis, leukopenia, neutropenia, and thrombocytopenia.

Given the inherent side effects associated with MTX use, there exists a need for further compounds with improved efficacy for treating neoplasfic, hyperproliferafive, and immune disorders, including cancer, arthritis, and psoriasis.

### SUMMARY OF THE INVENTION

Compositions and methods are provided for the inhibition of DHFR and the receptor polypeptides Her2, EGFR, and B-Raf. The Her2, EGFR, and B-Raf receptors are involved in cell proliferation, survival, and gene expression and have been implicated in neoplasia and the development of metastases. Consequently, these receptors are targets for antimetastatic and antiproliferative therapy, and have been implicated in other disorders as well. For instance, the EGFR system has also been implicated in proliferative and inflammatory diseases, including psoriasis and rheumatoid arthritis.

The present compositions also have reduced affinity for off-target polypeptides such as pyruvate carboxylase (PYC), propionyl-CoA carboxylase subunit alpha (PCCA), and propionyl-CoA carboxylase subunit beta (PCCB). These enzymes are involved in key metabolic pathways, and defects in their expression and/or activity cause deleterious consequences as described above. The identification of compounds with reduced affinity for pyruvate carboxylase and propionyl-CoA carboxylase offers alternatives to the use of compounds having the adverse effects of MTX.

Thus, the compositions and methods of the present invention offer improved treatments of neoplastic, hyperproliferative, and immune disorders.

According to one embodiment, there is provided a group of MTX derivatives.

According to another embodiment, there are provided pharmaceutical compositions comprising at least one compound from a group of MTX derivatives.

According to another embodiment, there are provided methods and compositions for inhibiting the activity of Her2, EGFR, and B-Raf.

According to another embodiment, there are provided methods and compositions for inhibiting the proliferation of a cell.

In another embodiment are provided methods and compositions for inhibiting the proliferation of a cell in a mammal.

According to another embodiment, there are provided methods and compositions for screening for additional folate antagonists utilizing the present compounds as a control.

According to another aspect of the present invention, there are provided methods of treatment of neoplastic, hyperproliferative, and immune disorders, including cancer and arthritis comprising administering at least one compound from a group of MTX derivatives.

According to yet another embodiment of the present invention, there is provided a method of preparation of MTX derivatives.

### DETAILED DESCRIPTION OF THE INVENTION

The present inventions now will be described more fully hereinafter with reference to specific embodiments of the invention. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

According to the present invention, there are provided novel compounds according to Formula II: 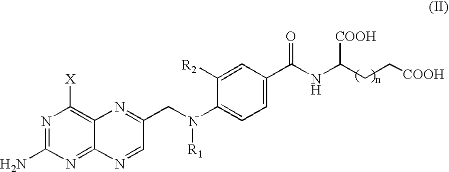
wherein:

X is OH or NH₂;
n=1 or 2;
R₁ is selected from the group consisting of alkenyl, alkynyl, cycloalkylalkyl, heteroarylalkyl, arylalkyl, C₅-C₁₀ alkyl; and
R₂ is selected from the group consisting of H, C₁-C₈ alkyl, CF₃, CF₃O, alkoxy, alkenyl, aralkyl (arylalkyl), heteroarylalkyl, alkynyl, CHO, COCH₃, and halogen;
provided that R₁ is C₅-C₁₀ alkyl when R₂ is H;
and pharmaceutically acceptable esters, amides, salts, or solvates thereof.

In another embodiment, there are provided novel compounds according to Formula II, wherein
X is OH or NH₂;
R₁ is selected from the group consisting of alkenyl, alkynyl, cyclopropylmethyl, heteroarylalkyl, arylalkyl, C₅-C₁₀ alkyl; and
R₂ is selected from the group consisting of C₁-C₈ alkyl, CF₃, CF₃O, alkoxy, aralkyl (arylalkyl), heteroarylalkyl, alkynyl, CHO, COCH₃, and halogen;
and pharmaceutically acceptable esters, amides, salts, or solvates thereof.

In another embodiment, there are provided novel compounds according to Formula II, wherein
X is NH₂;
R₁ is selected from the group consisting of alkenyl, alkynyl, cyclopropylmethyl, heteroarylalkyl, arylalkyl, C₅-C₁₀ alkyl; and
R₂ is selected from the group consisting of C₅-C₈ alkyl, CF₃O, alkoxy, aralkyl (arylalkyl), heteroarylalkyl, alkynyl, CHO, COCH₃, and halogen;
and pharmaceutically acceptable esters, amides, salts, or solvates thereof.

In another embodiment, there are provided novel compounds according to Formula II, wherein
X is OH or NH₂;
R₁ is selected from the group consisting of propargyl, furanylmethyl, cyclopropylmethyl, neopentyl, prenyl; and
R₂ is selected from the group consisting CF₃O, CH₃O, CF₃, CH₃, CH₃CH₂, C≡C—H, OH;
and pharmaceutically acceptable esters, amides, salts, or solvates thereof.

The present invention includes all stereoisomers of the compounds of Formula II either individually or admixed in any proportions. The stereoisomers of these compounds may include, but are not limited to, enantiomers, diastereomers, racemic mixtures and combinations thereof. Such stereoisomers can be prepared and separated using conventional techniques, either by reacting enantiomeric starting materials, or by separating isomers of compounds of the present invention. Isomers may include geometric isomers. Examples of geometric isomers include, but are not limited to, cis isomers or trans isomers across a double bond. Other isomers are contemplated among the compounds of the present invention. The isomers may be used either in pure form or in admixture with other isomers of the compounds described above.

The present invention further includes prodrugs and active metabolites of the compounds of Formula II. A prodrug includes any compound which, when administered to a mammal, is converted in whole or in part to a compound of Formula II. An active metabolite is a physiologically active compound which results from the metabolism of a compound of Formula II, or a prodrug thereof, when such compound or prodrug is administered to a mammal.

The compounds of Formula H above and their pharmaceutically acceptable esters, amides, salts, or solvates are sometimes hereinafter referred to as “the compounds according to the invention”.

The term “alkenyl” as used herein is intended to mean straight or branched chain unsaturated aliphatic hydrocarbons having one or more double bonds.

The term “alkynyl” as used herein is intended to mean straight or branched chain unsaturated aliphatic hydrocarbons having one or more triple bonds.

The term “alkyl” as used herein is intended to mean straight or branched chain alkyl.

The term “aryl,” alone or in combination, is intended to mean a monocyclic or polycyclic aromatic group.

The term “cycloalkyl” as used herein is intended to include monocyclic or fused polycyclic C₃-C₁₀ aliphatic hydrocarbon groups.

The term “haloalkyl” as used herein is intended to mean an alkyl group substituted with one or more halo substituents, either F, Cl, Br, or I, or combinations thereof.

The term “halogen” as used herein is intended to mean F, Cl, Br, or I.

The term “heteroaryl” as used herein is intended to mean a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, aryl, haloaryl, nitro, amino, alkylamino, acylamino, alkylthio, alkylsulfinyl, and alkylsulfonyl, arylsulfonyl, cyano.

The term “propargyl” as used herein is intended to mean R—CδC—CH₂—, wherein R is hydrogen, lower alkyl, haloalkyl, cycloalkyl, substituted or unsubstituted aryl, or substituted or unsubstituted heteroaryl.

The term “effective amount” as used herein is intended to mean an amount of a compound sufficient to achieve the desired effect, such as inhibiting the activity of a polypeptide, inhibiting the proliferation of a cell, modulating cell proliferation, survival, gene expression, cytostasis, cytotoxicity, tumor growth, or achieving an antimetastatic, antiproliferative or antiinflammatory effect. Methods of determining effective amounts are referenced herein below and are known in the art.

In one embodiment, a compound according to the present invention is according to Formula III: 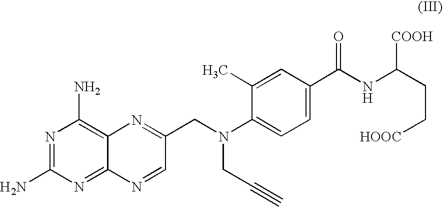
and pharmaceutically acceptable esters, amides, salts, or solvates thereof.

In another embodiment, a compound according to the present invention is according to Formula IV: 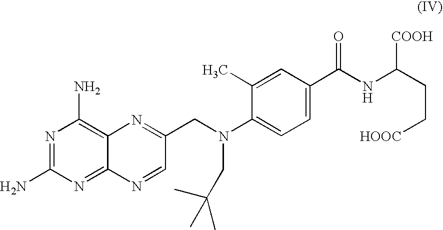
and pharmaceutically acceptable esters, amides, salts, or solvates thereof.

In another embodiment, a compound according to the present invention is according to Formula V: 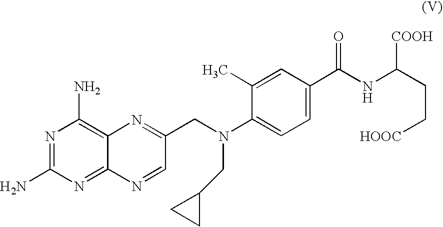
and pharmaceutically acceptable esters, amides, salts, or solvates thereof.

In another embodiment, a compound according to the present invention is according to Formula VI: 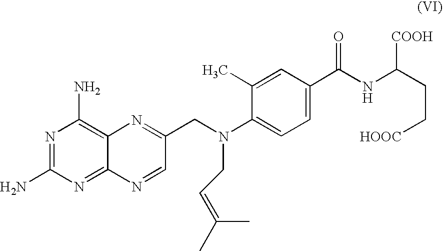
and pharmaceutically acceptable esters, amides, salts, or solvates thereof.

In another embodiment, a compound according to the present invention is according to Formula VII: 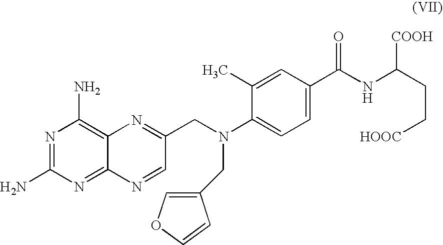
and pharmaceutically acceptable esters, amides, salts, or solvates thereof.

In another embodiment, a compound according to the present invention is according to Formula VIII: 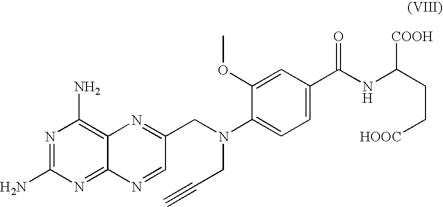
and pharmaceutically acceptable esters, amides, salts, or solvates thereof.

In another embodiment, a compound according to the present invention is according to Formula IX: 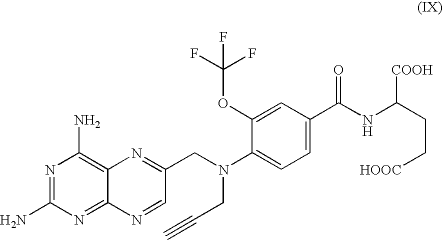
and pharmaceutically acceptable esters, amides, salts, or solvates thereof.

In another embodiment, a compound according to the present invention is according to Formula X: 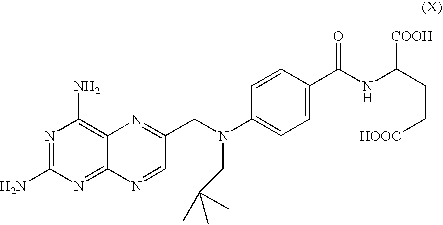
and pharmaceutically acceptable esters, amides, salts, or solvates thereof.

The present invention further provides processes for the preparation of MTX derivatives and esters, amides, salts, or solvates thereof. MTX derivatives generally provided in Formula II and their esters, amides, salts, and solvates may be prepared in any manner known in the art for the preparation of compounds of analogous structure. In particular, said compounds can be prepared according to the methods described herein. Stereoisomers of said compounds can be prepared and separated using conventional techniques, either by reacting enantiomeric starting materials, or by separating isomers of compounds of the present invention. With reference to the glutamic acid moiety for instance, a non racemic product can be obtained by synthesizing the compound using either D- or L-glutamic acid as a starting material. As is known to those of skill in the art, D- or L-glutamic acid are commercially available at various degrees of enantiopurity. By non-racemic is intended an end product in which one enantiomer is present in an amount greater than the other enantiomer. Alternatively, a racemic end product can be obtained by utilizing a racemic mixture of glutamic acid can be utilized as a starting material. A non-limiting exemplary method for the synthesis of compounds of Formula II is as follows.

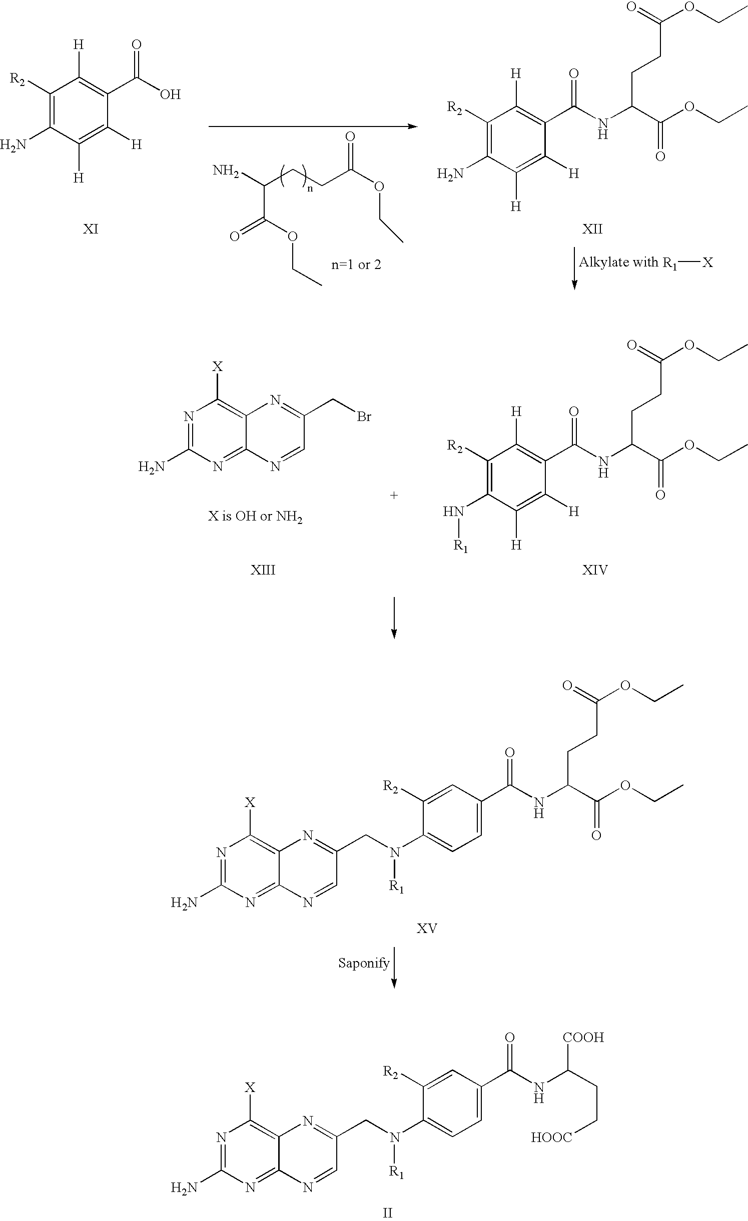

The compounds described herein can be administered in the form of an ester, amide, salt, solvate, prodrug, metabolite, derivative, or the like, provided it maintains pharmacological activity according to the present invention. Esters, amides, salts, solvates, prodrugs, and other derivatives of the compounds of the present invention may be prepared according to methods generally known in the art, such as, for example, those methods described by J. March, Advanced Organic Chemistry: Reactions, Mechanisms and Structure, 4^{th} Ed. (New York: Wiley-Interscience, 1992).

Examples of pharmaceutically acceptable salts of the compounds according to the invention include acid addition salts. Salts of non-pharmaceutically acceptable acids, however, may be useful, for example, in the preparation and purification of the compounds. Suitable acid addition salts according to the present invention include organic and inorganic acids. Preferred salts include those formed from hydrochloric, hydrobromic, sulfuric, phosphoric, citric, tartaric, lactic, pyruvic, acetic, succinic, fumaric, maleic, oxaloacetic, methanesulfonic, ethanesulfonic, p-toluenesulfonic, benzesulfonic, and isethionic acids. Other useful acid addition salts include propionic acid, glycolic acid, oxalic acid, malic acid, malonic acid, benzoic acid, cinnamic acid, mandelic acid, salicylic acid, and the like.

An acid addition salt may be reconverted to the free base by treatment with a suitable base. Preparation of basic salts of acid moieties which may be present on a compound of the present invention may be prepared in a similar manner using a pharmaceutically acceptable base, such as sodium hydroxide, potassium hydroxide, ammonium hydroxide, calcium hydroxide, triethylamine, or the like.

Esters of the compounds of the present invention may be prepared through functionalization of hydroxyl and/or carboxyl groups that may be present within the molecular structure of the compound. Amides and prodrugs may also be prepared using techniques known to those skilled in the art. For example, amides may be prepared from esters, using suitable amine reactants, or they may be prepared from anhydride or an acid chloride by reaction with ammonia or a lower alkyl amine. Prodrugs are typically prepared by covalent attachment of a moiety, which results in a compound that is therapeutically inactive until modified by an individual's metabolic system.

Inhibition of Her-2, EGFR, and B-Raf by the Compounds of the Invention

The compounds disclosed herein unexpectedly inhibit the Her-2, EGFR, and B-Raf receptor polypeptides with greater affinity than MTX. In particular, comparisons between a compound having the chemical structure of formula III, MTX, and the MTX derivative described as the “c series” in Piper et al. (1982) J. Med. Chem. 25:877-880 (herein after “the c series derivative”) revealed that the compound of Formula III displayed IC₅₀ values for Her2, EGFR and B-Raf of roughly 1-5 μM. Similar results have been obtained for compounds of Formula IV-X. MTX and the c series derivative displayed IC₅₀ values of roughly 10 μM or greater for these signaling molecules. See Example 2 and Tables 1 & 2, herein below, which describe the experimental work and results in detail.

By “binding affinity” is intended the strength of the interaction between two molecules, e.g., a compound and a polypeptide, two polypeptides, a polypeptide and a ligand, etc. By “bind” is intended an interaction between the two molecules measurable by techniques known in the art. Binding does not require an irreversible interaction between the molecules. Any method known in the art may be used to determine the amount of the one molecule that binds to another under a given set of conditions. Both direct binding assays and competitive binding assays can be used in a variety of different formats. For a description of different formats for binding assays, including competitive binding assays and direct binding assays, see, for example, Seethala and Femades, eds. (2001) Handbook of Drug Screening (Marcel Dekker, New York); and Mei and Czarnik, eds. (2002) Integrated Drug Discovery Technologies (Marcel Dekker, New York), both of which are herein incorporated by reference in their entirety for all purposes. Binding affinity of a compound for polypeptide may be measured by utilizing a competitive assay between the compound and polypeptide/ligand pair. For instance, binding affinity of a compound for polypeptide can be determined by assessing the IC₅₀ value for the interaction. By “IC₅₀” is intended the concentration of a compound required to inhibit the binding of a ligand by 50%.

For instance, the IC₅₀ value for DHFR can be determined by enzyme assay using purified DHFR (Rosowsky et al. (1991) J. Med. Chem. 34:1447-54). Alternatively, IC₅₀ values can be estimated empirically by Proteome Mining™ (see WO 00/63694) or determined by Affinity Displacement Avidity Effect Protein Resolution Methods (U.S. Provisional Application No. 60/532,122), each of which is incorporated herein in their entirety.

Her-2, also known as ErbB-2 or c-neu (Bargmann et al. (1986) Nature 319:226-230), and EGFR are both tyrosine kinase receptor polypeptides of the Epidermal Growth Factor (EGF) family. Her-2 and EGFR are expressed in a wide variety of cell types (Kraus et al. (1989) PNAS USA 86:9193-9197). Activation of the receptor and signaling occurs when ligand binding results in receptor dimerization. Her2 is the preferred dimerization partner for EGFR (Janmaat et al. (2003) The Oncologist 8:576-586). These receptors can also transmit signals to cells upon truncation or mutation, or upon amplification of basal receptor activity (without ligand) through cooperation with other cellular signaling pathways or nuclear events. Aberrant EGFR expression or activation can, among other effects, contribute to neoplasia and development of metastases (Khazaie et al. (1993) Cancer Metastasis Rev. 12:255-274). EGFR oncogenic potential has been demonstrated in a wide range of animal models and, in humans, is implicated in the initiation (glioblastoma) and progression (epithelial tumors) of the disease. Gene amplification/overexpression of either the EGFR or Her-2 associated with their constitutive activation has been observed in a wide variety of human tumors (Khazaie et al. (1993) Cancer Metastasis Rev. 12:255-274; Dougall et al. (1994) Oncogene 9:2109-2123). Consequently, these receptors are targets for antimetastatic therapy. Beyond its role in oncogenesis, the EGFR system has also been implicated in other proliferative diseases, including psoriasis (Ben-Bassat et al. (2000) Curr. Pharm. Des. 6:933-42), as well as inflammatory disorders such as rheumatoid arthritis (Lui et al. (2002) J. Cell. Physiol. 192:102-12).

One important signaling route activated by the EGFR involve the Ras-Raf-MEK-ERK kinase pathways, which are implicated in cell proliferation, survival, and gene expression (Janmaat et al. (2003) The Oncologist 8:576-586). B-Raf is one of three known mammalian Raf isoforms. Raf is a serine-threonine kinase polypeptide (Kolch et al. (1991) Nature 349:426-428). Ras has been shown to be oncogenically activated by mutations in over 15% of all human tumors (Bos (1989) Cancer Res. 49:4682-4689). While A-Raf is ubiquitously expressed, B-Raf is highly restricted to neural-derived tissues (Lewis et al. (1998) Adv. Cancer Res. 74:49-139; Rapp et al. (1988) Cold Spring Harb. Symp. Quant. Biol. 53:173-184). Once activated, Raf can phosphorylate MEK leading to its activation (Lewis et al. (1998) Adv. Cancer Res. 74:49-139). Mutationally activated forms of Raf or MEK can transform rodent fibroblasts and form tumors in nude mice (Campbell et al. (1998) Oncogene 17:1395-1413). In vivo experiments have co-localized activated EGFR, Ras, and a c-Raf fusion protein in endosomal compartments (Sorkin et al. (2002) Mol. Biol. Cell 13:1522-1535). Given its participation in Ras signaling and its implication in cellular transformation, B-Raf is a target for anticancer therapy.

Thus, methods and compositions are provided to inhibit Her2, EGFR, and B-Raf activity using the compounds of the invention. In one embodiment, the method comprises the steps of contacting at least one of the Her2, EGFR, and B-Raf polypeptides with an amount of the compound of the invention, determining Her2, EGFR, and B-Raf activity, and comparing the activity determined to that of the polypeptide in the absence of the compound. A decrease in the activity in the presence of the compound is indicative of inhibition.

By “Her2-”, “EGFR-”, or “B-Raf-activity” is intended mechanisms resulting in any of the art-recognized effects associated with these molecules. The effects associated with these molecules can be assessed by standard techniques known in the art, including binding assays, immuno-assays, expression assays, cellular proliferation assays, etc. It is understood that the decrease in “Her2-”, “EGFR-”, or “B-Raf-activity” is represented by modulation of the effect measured by the assay. The modulation can be either an increase or a decrease in the effect measured by the assay. For instance, inhibition of EGFR activity favors several proapoptotic mechanisms, such as the activation of the proapoptotic protein BAD or upregulation of p27^{kip1}. Thus, the decrease in EGFR activity leads to an increase in a measurable cellular effect. On the other hand, the modulation may be a decrease in kinase activity or other downstream signaling. Assays for “Her2-”, “EGFR-”, or “B-Raf-activity” are known in the art. See, for instance, Fiorentino et al. (2000) Mol. Cell. Biol. 207735-7750 and Dougall et al. (1994) Oncogene 9:2109-23 (Her2 assays); Albanell et al. (2002) J. Clin. Oncol. 20:110-124, Albanell et al. (2001) Semin. Oncol. 28:56-66 (EGFR assays); Reuter et al. (1995) Methods Enzymol. 255:245-56 and Ikenoue et al. (2003) Cancer Res. 63:8132-7 (B-Raf assays). Kits for such assays can be purchased commercially. See, for example, the Protein Tyrosine Kinase Assay Kit from Sigma™.

In a further embodiment, the compound is selected from the class of compounds set forth in Formula II. In another embodiment, the compound is selected from the class of compounds set forth in Formula II wherein the R₁ substituent is 2-propyne. In yet a further embodiment, the compound is a compound set forth in Formulas III-X.

In another embodiment, the compound inhibits Her2, EGFR, and B-Raf. In another embodiment, the compound inhibits at least one of Her2, EGFR, and B-Raf.

In a further embodiment, the inhibition by the present compounds leads to a measurable change in activity that is double or more than double that determined for MTX. For instance the decrease in activity may be 2, 3, 4, 5, 6, 7, 8, 9, 10, 25, 50, 100, 500, 1000, or 10,000-fold that of MTX.

In another embodiment, compounds that have a binding affinity for Her2, EGFR, and B-Raf greater than the binding affinity of MTX for Her2, EGFR, and B-Raf are provided. In another embodiment, compounds that have a binding affinity for at least one of Her2, EGFR, or B-Raf greater than the binding affinity of MTX for at least one of Her2, EGFR, and B-Raf are provided. In a further embodiment, the binding affinity of the compound is more than double that determined for MTX. For instance, binding affinity may be 2, 3, 4, 5, 6, 7, 8, 9, 10, 25, 50, 100, 500, 1000, or 10,000-fold that of MTX.

Antiproliferative Activity of the Compounds of the Invention

The compounds of the invention demonstrate improved antiproliferative activity when compared to MTX. See Example 3 and Tale 3. Thus, methods for inhibition of proliferation and compositions with antiproliferative activity are provided. “Antiproliferative activity” refers to the ability of a compound to induce cytostasis or cytotoxicity. “Cytostasis” is the inhibition of cells from growing while “cytotoxicity” is defined as the killing of cells. “Inhibition of proliferation” refers to the results of antiproliferative activity.

Inhibition of proliferation and antiproliferative activity can be assessed in vitro by any means known in the art. For instance, an assay for cell proliferative activity can be utilized. Proliferative activity assays include those that assess metabolic activity, DNA synthesis, apoptosis, necrosis, telomerase activity, etc. It is recognized in the art that the efficacy of therapeutic agents in drug screening and the cytostatic potential of anticancer compounds in toxicology testing can be assessed when quantifying inhibition of proliferation or antiproliferative activity in vitro.

Nonlimiting examples of assays for proliferative activity include those that monitor metabolic activity by cellular tetrazolium salt cleavage (Mosmann (1983) J. Immunol. Methods 65:55; Mosmann et al. (1983) J. Immunol. Methods 116:151); DNA synthesis assays that monitor BrdU incorporation (Hall et al. (1990) J. Clin. Pathol. 43:184; Hall et al. (1990) J. Pathol. 162:285); and the annexin V apoptosis assay for membrane integrity (Hoornaert et al. (1997) Biochemica 3:19-20).

Inhibition of proliferation and antiproliferative activity can be quantitated by measuring the cell proliferation inhibition IC₅₀ using any assay for proliferative activity. See Example 4, comparing the cell proliferation inhibition IC₅₀ values for MTX, the c series derivative, and the presently disclosed compounds. By “cell proliferation inhibition IC₅₀” is intended the concentration of a test compound causing 50% inhibition of cell proliferation in a population of cells contacted with the test compound, as compared to a control population of cells not contacted with the test compound.

In one embodiment, the method for inhibiting proliferation comprises the steps of contacting a cell with an amount of a compound of the invention, measuring the proliferative activity of the cell, and comparing the activity to that of the cell in the absence of the compound. A decrease in proliferative activity is indicative of inhibition of proliferation. In one embodiment, the cell proliferation inhibition IC₅₀ value is used to measure proliferative activity.

In a further embodiment, the compound is selected from the class of compounds set forth in Formula II. In another embodiment, the compound is selected from the class of compounds set forth in Formula II wherein the R₁ substituent is 2-propyne. In yet a further embodiment, the compound is the compound set forth in Formula III.

In another embodiment, the inhibition is twice or more than twice that determined for MTX. For instance the inhibition may be 2, 3, 4, 5, 6, 7, 8, 9, 10, 25, 50, 100, 500, 1000, or 10,000-fold that of MTX.

In another embodiment, compounds with antiproliferative activity are provided. In another embodiment, compounds with greater antiproliferative activity than MTX are provided. In a further embodiment, the antiproliferative activity is twice or more than twice that determined for MTX. For instance the antiproliferative activity may be 2, 3, 4, 5, 6, 7, 8, 9, 10, 25, 50, 100, 500, 1000, or 10,000-fold that of MTX.

Inhibition of proliferation and antiproliferative activity can also be assessed in vivo. For instance, inhibition of proliferation of a compound can be tested in vivo on primary tumors by, for example, implanting human tumor cells subcutaneously in athymic mice. Exemplary human tumor cell lines which can be used include, but are not limited to prostate carcinoma (PC3 cells), colon adenocarcinoma (HCT29 cells), and mammary adenocarcinoma (MCF-7 cells). Treatment with control solution or a test compound of the invention begins when tumors are approximately 100 mg. Anti-tumor activity is assessed by measuring the delay in tumor growth, and/or tumor shrinking and/or increased survival of the treated animals relative to control animals.

Selectivity of the Compounds of the Invention

In contrast to the presently disclosed compounds' greater inhibition of Her2, EGFR, and B-Raf as compared to MTX, the present compounds display reduced binding affinity values for pyruvate carboxylase and propionyl-CoA carboxylase subunits A and B. See Example 2 and Table 2.

Pyruvate carboxylase and propionyl-CoA carboxylase subunits A and B undesireably interact with MTX with IC₅₀ values of 0.35 μM, 0.85 μM, and 0.85 μM, respectively. The compound having the chemical structure of Formula III displayed IC₅₀ values of 3.8 μM, 2 μM, and 3.5 μM with PYC, PCCA, and PCCB, respectively. The c series derivative displayed IC₅₀ values of 0.66 μM, 2 μM, and 3.5 μM with the same polypeptides.

Pyruvate carboxylase (EC 6.4.1.1) catalyzes the ATP-dependent carboxylation of pyruvate:
ATP+pyruvate+HCO₃⁻=ADP+phosphate+oxaloacetate

Pyruvate carboxylase is a key regulatory enzyme in gluconeogenesis, lipogenesis, and neurotransmitter synthesis. The human amino acid sequence for pyruvate carboxylase is described by Freytag and Collier (1984) J. Biol. Chem. 259:12831-37 and is given in Swiss-Prot accession number JC2460, both of which are herein incorporated by reference. Two distinct clinical presentations of pyruvate deficiency have been identified. An infantile form present soon after birth with chronic lacticacidemia, and delayed neurologic development in survivors. The second form also presents early with lactic acidosis but shows elevated blood levels of ammonia, citrulline, proline, and lysine. Mutations in the human pyruvate carboxylase gene that result in pyruvate carboxylase deficiency have been identified (Carbone et al. (1998) Am. J. Hum. Genet. 62:1312-19; Wexler et al. (1998) Pediat. Res. 43:579-84; and Carbone et al. (2002) Hum. Mutat. 20:48-56). By “pyruvate carboxylase” as used herein, it is intended an enzyme from enzyme class 6.4.1.1. In particular embodiments, the pyruvate carboxylase is mammalian pyruvate carboxylase, such as, for example human pyruvate carboxylase, although the pyruvate carboxylase may be from any source.

Propionyl-CoA carboxylase (EC 6.4.1.3) catalyzes the first step in the catabolism of propionyl-CoA:
ATP+propanoyl-CoA+HCO₃⁻=ADP+phosphate+(S)-methylmalonyl-CoA

This enzyme also carboxylates butanoyl-CoA and catalyzes transcarboxylation. Propionyl-CoA is an important intermediate in the metabolism of isoleucine, threonine, methionine, and valine. Propionyl-CoA carboxylase is composed of two non-identical subunits, alpha (PCCA) and beta (PCCB). The human amino acid sequences for PCCA and PCCB are described by Lamhonwah et al. (1986) Proc. Natl. Acad. Sci. U.S.A. 83:4864-4868 and are given in Swiss-Prot accession numbers PO₅₁₆₅ (PCCA) and PO₅₁₆₆ (PCCB), each of which is herein incorporated by reference. Mutations in the human PCCA and PCCB gene are responsible for propionic acidemia, an autosomal recessive disease characterized by an excess of propionic acid in the blood and urine, with ketosis, acidosis, hyperglycinemia, hyperglycinuria, and often neurologic complications (Lamhonwah et al. (1986) Proc. Natl. Acad. Sci. U.S.A. 83:4864-4868). By “propionyl-CoA carboxylase” as used herein, it is intended an enzyme from enzyme class 6.4.1.3, or a subunit of such an enzyme (e.g., PCCA or PCCB). In particular embodiments, the propionyl-CoA carboxylase is mammalian propionyl-CoA carboxylase, such as, for example human propionyl-CoA carboxylase, although the propionyl-CoA carboxylase may be from any source.

The use of MTX in therapy for neoplastic, hyperproliferative, and immune disorders is associated with a number of adverse effects, particularly when MTX is used a higher dosages. MTX-associated adverse effects include alopecia, dermatitis, interstitial pneumonitis, nephrotoxicity, defective oogenesis or spermatogenesis, abortion, and teratogenesis, hepatotoxicity, hepatic dysfunction, cirrhosis, and damage to the central nervous system.

The identification of compounds with reduced affinity for pyruvate carboxylase and propionyl-CoA carboxylase offers alternatives to the use of compounds having the adverse effects of this class of drugs. These enzymes are involved in key metabolic pathways, and defects in their expression and/or activity cause deleterious consequences as described above.

Thus, the compounds of the invention can be utilized to screen for additional compounds having improved binding selectivity, improved Her2, EGFR, and B-Raf inhibition, and/or improved antiproliferative activity as compared to the compounds of the invention. By “binding selectivity” it is intended the degree to which the folate antagonist binds the folate-dependent enzyme relative to other proteins in cell, particularly pyruvate carboxylase and propionyl-CoA carboxylase.

In one embodiment, the methods comprise screening one or more compounds of interest to determine whether the compound or compounds possess improved selectivity.

The compound of interest may be selected from a group of compounds with similar structure to the present compounds, or may be selected because it possesses an activity of interest, such as acting as a folate antagonist. For instance, the compound of interest can be a known folate antagonist or, alternately, the compound can be screened to determine whether it is a folate antagonist using methods known to those of skill in the art. Further methods of screening folate antagonists are disclosed in U.S. Provisional Application No. 60/515,012, which is incorporated herein in its entirety.

Compounds of interest are screened to determine the level of binding to at least one enzyme selected from pyruvate carboxylase, propionyl-CoA carboxylase, or a subunit thereof, and comparing the values determined for the compound or compounds of interest to those for the compounds disclosed herein. A compound that has a lower level of binding to one or more enzymes selected from pyruvate carboxylase and propionyl-CoA carboxylase, or a subunit thereof, is identified as a compound having increased selectivity. A “low level of binding” to pyruvate carboxylase or propionyl-CoA carboxylase, or a subunit thereof according to the invention is a level of binding that is lower than that observed under the same binding conditions for the compounds disclosed herein. In some embodiments, the compound having increased selectivity has a significantly decreased risk of adverse effects in treatment.

In another embodiment, the methods comprise screening one or more compounds of interest to determine the level of binding of the compound or compounds to at least one polypeptide selected from Her2, EGFR, and B-Raf, determining the degree to which the compound of interest inhibits at least one of Her2-, EGFR-, and B-Raf-activity, and comparing the values determined for the compound of interest to those obtained for the compounds disclosed herein. An “improved inhibition” of Her2, EGFR, and/or B-Raf according to the invention is a level of inhibition that is higher than that observed under the same binding conditions for the compounds disclosed herein. A compound of interest that has a higher level of binding to one or more polypeptides selected from Her2, EGFR, and B-Raf is identified as a compound having improved inhibition of Her2, EGFR, and/or B-Raf activity.

In another embodiment, the methods comprise screening one or more compounds of interest to determine the level of antiproliferative activity, and comparing the values determined for the compound of interest to those obtained for the compounds disclosed herein.

In yet another embodiment, the methods comprise the combination of screening one or more compound of interest for improved binding selectivity, improved Her2, EGFR, and B-Raf inhibition, and/or improved antiproliferative activity as compared to the compounds of the invention.

Formulations

While it is possible for the compounds of the present invention to be administered in the raw chemical form, it is preferred for the compounds to be delivered as a pharmaceutical formulation. Accordingly, there are provided by the present invention pharmaceutical compositions comprising at least one compound from a group of MTX derivatives. As such, the formulations of the present invention comprise a compound of Formula II, as described above, or a pharmaceutically acceptable ester, amide, salt, or solvate thereof, together with one or more pharmaceutically acceptable carriers therefore, and optionally, other therapeutic ingredients.

By “pharmaceutically acceptable carrier” is intended a carrier that is conventionally used in the art to facilitate the storage, administration, and/or the healing effect of the agent. Carriers should be acceptable in that they are compatible with any other ingredients of the formulation and not harmful to the recipient thereof. A carrier may also reduce any undesireable side effects of the agent. Such carriers are known in the art. See, Wang et al. (1980) J. Parent. Drug Assn. 34(6):452-462, herein incorporated by reference in its entirety.

Formulations of the present invention may include short-term, rapid-onset, rapid-offset, controlled release, sustained release, delayed release, and pulsatile release formulations, providing the formulations achieve administration of a compound as described herein. See Remington's Pharmaceutical Sciences (18^{th} ed.; Mack Publishing Company, Eaton, Pa., 1990), herein incorporated by reference in its entirety.

Pharmaceutical formulations according to the present invention are suitable for various modes of delivery, including oral, parenteral (including intravenous, intramuscular, subcutaneous, intradermal, and transdermal), topical (including dermal, buccal, and sublingual), and rectal administration. The most useful and/or beneficial mode of administration can vary, especially depending upon the condition of the recipient and the disorder being treated.

The pharmaceutical formulations may be conveniently made available in a unit dosage form, whereby such formulations may be prepared by any of the methods generally known in the pharmaceutical arts. Generally speaking, such methods of preparation comprise combining (by various methods) an active agent, such as the compounds of Formula II according to the present invention (or a pharmaceutically acceptable ester, amide, salt, or solvate thereof) with a suitable carrier or other adjuvant, which may consist of one or more ingredients. The combination of the active ingredient with the one or more adjuvants is then physically treated to present the formulation in a suitable form for delivery (e.g., shaping into a tablet or forming an aqueous suspension).

Pharmaceutical formulations according to the present invention suitable as oral dosage may take various forms, such as tablets, capsules, caplets, and wafers (including rapidly dissolving or effervescing), each containing a predetermined amount of the active agent. The formulations may also be in the form of a powder or granules, a solution or suspension in an aqueous or non-aqueous liquid, and as a liquid emulsion (oil-in-water and water-in-oil). The active agent may also be delivered as a bolus, electuary, or paste. It is generally understood that methods of preparations of the above dosage forms are generally known in the art, and any such method would be suitable for the preparation of the respective dosage forms for use in delivery of the compounds according to the present invention.

A tablet containing a compound according to the present invention may be manufactured by any standard process readily known to one of skill in the art, such as, for example, by compression or molding, optionally with one or more adjuvant or accessory ingredient. The tablets may optionally be coated or scored and may be formulated so as to provide slow or controlled release of the active agent.

Adjuvants or accessory ingredients for use in the formulations of the present invention can include any pharmaceutical ingredient commonly deemed acceptable in the art, such as binders, fillers, lubricants, disintegrants, diluents, surfactants, stabilizers, preservatives, flavoring and coloring agents, and the like. Binders are generally used to facilitate cohesiveness of the tablet and ensure the tablet remains intact after compression. Suitable binders include, but are not limited to: starch, polysaccharides, gelatin, polyethylene glycol, propylene glycol, waxes, and natural and synthetic gums. Acceptable fillers include silicon dioxide, titanium dioxide, alumina, talc, kaolin, powdered cellulose, and microcrystalline cellulose, as well as soluble materials, such as mannitol, urea, sucrose, lactose, dextrose, sodium chloride, and sorbitol. Lubricants are useful for facilitating tablet manufacture and include vegetable oils, glycerin, magnesium stearate, calcium stearate, and stearic acid. Disintegrants, which are useful for facilitating disintegration of the tablet, generally include starches, clays, celluoses, algins, gums, and crosslinked polymers. Diluents, which are generally included to provide bulk to the tablet, may include dicalcium phosphate, calcium sulfate, lactose, cellulose, kaolin, mannitol, sodium chloride, dry starch, and powdered sugar. Surfactants suitable for use in the formulation according to the present invention may be anionic, cationic, amphoteric, or nonionic surface active agents. Stabilizers may be included in the formulations to inhibit or lessen reactions leading to decomposition of the active agent, such as oxidative reactions.

Solid dosage forms may be formulated so as to provide a delayed release of the active agent, such as by application of a coating. Delayed release coatings are known in the art, and dosage forms containing such may be prepared by any known suitable method. Such methods generally include that, after preparation of the solid dosage form (e.g., a tablet or caplet), a delayed release coating composition is applied. Application can be by methods, such as airless spraying, fluidized bed coating, use of a coating pan, or the like. Materials for use as a delayed release coating can be polymeric in nature, such as cellulosic material (e.g., cellulose butyrate phthalate, hydroxypropyl methylcellulose phthalate, and carboxymethyl ethylcellulose), and polymers and copolymers of acrylic acid, methacrylic acid, and esters thereof.

Solid dosage forms according to the present invention may also be sustained release (i.e., releasing the active agent over a prolonged period of time), and may or may not also be delayed release. Sustained release formulations are known in the art and are generally prepared by dispersing a drug within a matrix of a gradually degradable or hydrolyzable material, such as an insoluble plastic, a hydrophilic polymer, or a fatty compound. Alternatively, a solid dosage form may be coated with such a material.

Formulations for parenteral administration include aqueous and non-aqueous sterile injection solutions, which may further contain additional agents, such as anti-oxidants, buffers, bacteriostats, and solutes, which render the formulations isotonic with the blood of the intended recipient. The formulations may include aqueous and non-aqueous sterile suspensions, which contain suspending agents and thickening agents. Such formulations for patenteral administration may be presented in unit-dose or multi-dose containers, such as, for example, sealed ampoules and viles, and may be stores in a freeze-dried (lyophilized) condition requiring only the addition of the sterile liquid carrier, for example, water (for injection), immediately prior to use. Extemporaneous injection solutions and suspensions may be prepared from sterile powders, granules, and tablets of the kind previously described.

The compounds according to the present invention may also be administered transdermally, wherein the active agent is incorporated into a laminated structure (generally referred to as a “patch”) that is adapted to remain in intimate contact with the epidermis of the recipient for a prolonged period of time. Typically, such patches are available as single layer “drug-in-adhesive” patches or as multi-layer patches where the active agent is contained in a layer separate from the adhesive layer. Both types of patches also generally contain a backing layer and a liner that is removed prior to attachment to the skin of the recipient. Transdermal drug delivery patches may also be comprised of a reservoir underlying the backing layer that is separated from the skin of the recipient by a semi-permeable membrane and adhesive layer. Transdermal drug delivery may occur through passive diffusion or may be facilitated using electrotransport or iontophoresis.

Formulations for rectal delivery of the compounds of the present invention include rectal suppositories, creams, ointments, and liquids. Suppositories may be presented as the active agent in combination with a carrier generally known in the art, such as polyethylene glycol. Such dosage forms may be designed to disintegrate rapidly or over an extended period of time, and the time to complete disintegration can range from a short time, such as about 10 minutes, to an extended period of time, such as about 6 hours.

Topical formulations may be in any form suitable and readily known in the art for delivery of an active agent to the body surface, including dermally, buccally, and sublingually. Typical examples of topical formulations include ointments, creams, gels, pastes, and solutions. Formulations for topical administration in the mouth also include lozenges.

Preferred unit dosage formulations are those containing a therapeutically effective amount, or an appropriate fraction thereof, of the active agent of the present invention. The term therapeutically effective amount, as used herein, is meant to refer to an amount effective to treat the disease of interest, such as cancer or arthritis. Treatment can mean having a direct effect on an area in need of treatment, such as a tumor, or having a peripheral effect, such as through the activation or inhibition of a therapeutically associated enzyme.

Compounds are administered in amounts of 10-200 mg/m² of body surface area per day. For instance, compounds may be administered in amounts of 20-40 mg/m² of body surface area per day. For instance, compounds may be administered in amounts of 40-60 mg/m² of body surface area per day. For instance, compounds may be administered in amounts of 60-80 mg/m² of body surface area per day. For instance, compounds may be administered in amounts of 80-100 mg/m² of body surface area per day. For instance, compounds may be administered in amounts of 100-120 mg/m² of body surface area per day. For instance, compounds may be administered in amounts of 120-140 mg/m² of body surface area per day. For instance, compounds may be administered in amounts of 140-160 mg/m² of body surface area per day. For instance, compounds may be administered in amounts of 160-180 mg/m² of body surface area per day. For instance, compounds may be administered in amounts of 180-200 mg/m² of body surface area per day.

Also provided according to the present invention is the use of the compounds provided herein in medical therapy, particularly for the treatment of cancer and arthritis. In one embodiment according to the present invention, there is provided a method for treating cancer comprising administering to a patient in need thereof a therapeutically effective amount of a compound of Formula II as described above. In another embodiment according to the present invention, there is provided a method for treating arthritis comprising administering to a patient in need thereof a therapeutically effective amount of a compound of Formula II as described above.

Compounds of Formula II are useful in the treatment of diseases associated with aberrant neoplastic growth such as cancer and certain arthritic and inflammatory conditions. In particular they are useful in the treatment of breast cancer, head and neck cancer, lung cancer, acute lymphocytic leukemia, lymphomas, mesotheliomas, colon cancer, prostate cancer, melanoma, and inflammatory diseases such as Rheumatoid Arthritis, osteoarthritis, lupus and psoriasis.

The following Examples illustrate the present invention but should not be construed as a limitation to the scope thereof.

### EXAMPLE 1

### Preparation of 2-{4-[(2,4-diamino-pteridin-6-ylmethyl)-Prop-2-ynyl-amino]-3-methyl-benzoylamino}-pentanedioic acid

Initially, the compound 6-bromomethyl-pteridine-2,4-diamine as shown in Formula XIII 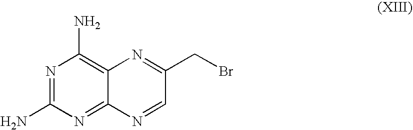
was prepared by first suspending 2,4-diamino-6-(hydroxymethyl)pteridine hydrochloride (2.29 g, 10 mmol) in 100 ml glacial acetic acid, warming to reflux and then allowing to cool to ambient temperature. Hydrobromic acid and 30% AcOH solution (191 ml) was then added and the flask stoppered for 4 days. The reaction mixture was then poured into 1200 ml dry diethyl ether with stirring. The product was allowed to settle and excess solvent was removed by decantation. The product was collected, washed with additional ether, and dried for later use.

2-(4-Amino-3-methyl-benzoylamino)-pentanedioic acid diethyl ester as shown in Formula XVI 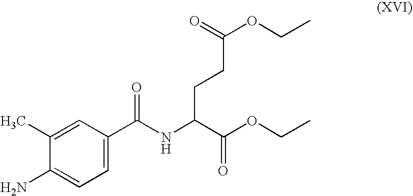
was prepared by first combining 1.51 g 4-amino-3-methylbenzoic acid (10 mmol), 2.60 g L-glutamic acid diethyl ester hydrochloride (10.5 mmol), 6 ml N,N-dimethylformamide, and 1.5 ml triethylamine (11 mmol). Next, 1.5 g 1-(3-dimethylaminopropyl)-3-ethylcarbodiimide hydrochloride (11 mmol) was added, and the reaction was stirred for 44 hours. The crude product was taken up in 200 ml ethyl acetate and washed with 100 ml water. The organic layer was dried over magnesium sulfate, concentrated, and collected as an oil.

The compound of Formula XVI (2.03 g, 6 mmol) was then diluted with 4 ml N,N-dimethylformamide and 1.22 ml diisopropylethylamine (7 mmol), and 0.73 ml 80 wt. % propargyl bromide in toluene (6.6 mmol) was added. The solution was stirred for three days, diluted with 200 ml ethyl acetate and washed with 100 ml water, and 1.01 g of propargylated aniline product (45%) was collected as an oil.

The propargylated aniline and the bromide of Formula XVI were suspended in N,N-dimethylacetamide and stirred in the dark for 4 days. The reaction mixture was then poured into a solution of 500 mg sodium bicarbonate in 100 ml water. The solid was collected, washing the frit with ethanol to ensure complete collection of the product. The solid and washings were concentrated and passed through a silica gel column, eluting with 8:2 chloroform:methanol. The resulting product, 2-{4-[(2,4-diamino-pteridin-6-ylmethyl-prop-2-ynyl-amino]-3-methyl-benzoylamino}-pentanedioic acid diethyl ester (214 mg, 14%), as shown in Formula XVII was then collected.

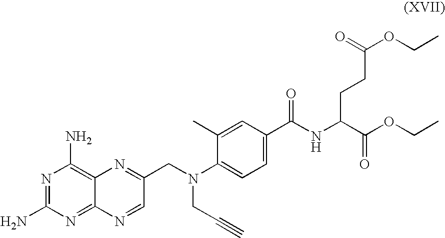

The compound of Formula XVII was then diluted with 4 ml ethanol and 4 ml water, and 1 ml NaOH (1 N) was added. TLC indicated complete saponification in 2 hours. The reaction mixture was concentrated to remove the ethanol and diluted with 10 ml additional water. The pH was adjusted to 4.0 using dilute HCl causing the product, 2-{4-[(2,4-diamino-pteridin-6-ylmethyl)-prop-2-ynyl-amino]-3-methyl-benzoylamino}-pentanedioic acid, as shown in Formula III, to precipitate from solution. The mixture was allowed to settle under refrigeration for about 20 minutes, and the product was collected on paper by suction filtration. The product was scraped into a flask, and the filter paper was washed with ethanol. Concentration in vacuo to constant weight afforded the desired pteridinyl diacid as a yellow solid (126 mg, 67%). NMR and mass spectrometry were consistent with expectations.

### EXAMPLE 2

### Determination of IC₅₀ Values for Her2, EGFR, B-Raf, PYC, PCCA, and PCCB

IC₅₀ values for compounds of Formulas III-X were estimated and compared to those for MTX and the c series derivative as follows. A proteome containing a known quantity of Her2, EGFR, and B-Raf was run over an ATP-Sepharose™ column in order to bind all purine-binding proteins. See, WO 00/63694 and U.S. Provisional Application No. 60/453,697 filed Jan. 22, 2003, each of which is incorporated by reference in its entirety. The ATP-Sepharose™ was washed several times, and then eluted with the compound of Formula III. The experiment was run in duplicate at concentrations of 20 μM, 100 μM, and 500 μM of the compound of Formula III. The eluted fractions were run on a 1-dimensional SDS polyacrylamide gel. Gels were stained with a fluorescent stain such as sypro ruby (a highly sensitive fluorescent protein stain that can readily detect less than 1 fmol of total protein, i.e., less than 0.04 ng for a 40 kDa protein). The gels were imaged using a standard flat bed gel imager and the amount of protein estimated by densiometry. The percent of protein eluted from the column at each concentration was determined and IC₅₀ values were calculated from these estimates. Where necessary, the eluted proteins were cut out of the gel, and identified using mass spectral techniques.

These steps were repeated with each of the compounds of Formulas IV-X, MTX, and the c series derivative. The values for each compound (where determined) are set forth in Table 1, below.

TABLE 1
Approximate IC₅₀ Values for Her2, EGFR, and B-Raf

| | IC₅₀ for Relevant | | |
|---|---|---|---|
| | Polypeptide (μM) | | |
| Compound | Her2 | EGFR | B-Raf |
| c Series Derivative | >10 | >10 | >10 |
| Formula III | 0.9 | 5 | 5 |
| Formula IV | n/d | n/d | n/d |
| Formula V | 5 | 10 | 10 |
| Formula VI | 10 | 15 | n/d |
| Formula VII | 5 | 10 | 10 |
| Formula VIII | 5 | 10 | 5 |
| Formula IX | 7.5 | 15 | n/d |
| Formula X | n/d | n/d | n/d |
| MTX | 10 | >15 | n/d |

n/d = not determined

Similar experiments were carried out to compare the IC₅₀ values for PYC, PCCA, and PCCB. In these trials, a porcine liver proteome was run over an ATP-Sepharose™ column to bind all purine-binding proteins. The ATP-Sepharose™ column was washed several times, and then eluted with the compound of Formula III at concentrations of 1 μM, 5 μM, 10 μM, 50 μM, 100 μM, and 500 μM to identify proteins that bound to this compound. (The amount of protein eluted at the higher concentration ranges plateaued, thus confirming that the entire fraction of bound protein had been eluted.) The eluted fractions were run on a 1-dimensional SDS polyacrylamide gel.

Gels were stained with a fluorescent stain such as sypro ruby (a highly sensitive fluorescent protein stain that can readily detect less than 1 fmol of total protein, i.e., less than 0.04 ng for a 40 kDa protein). The gels were imaged using a standard flat bed gel imager and the amount of protein estimated by densiometry. The percent of protein eluted from the column at each concentration was determined and IC₅₀ values were calculated from these estimates. Where necessary, the eluted proteins were cut out of the gel, and identified using mass spectral techniques.

These steps were repeated with each of the compounds of Formulas IV-X, MTX, and the c series derivative. The values obtained were then compared to those for DHFR. The values (where determined) are set forth in Table 2, below.

TABLE 2
Approximate IC₅₀ Values for PYC, PCCA, PCCB, and DHFR

| | IC₅₀ for Relevant Polypeptide in μM | | | | |
|---|---|---|---|---|---|
| | (or nM where indicated) | | | | |
| Compound | PYC | PCCA | PCCB | DHFR^{a} | |
| c Series Derivative | 0.66 | 2 | 3.5 | 18.8 | nM^{a} |
| Formula III | 3.8 | 2 | 3.5 | 10 | nM^{a} |
| Formula IV | 5 | 6 | 10 | 17 | nM^{a} |
| Formula V | 4 | 2 | 3.5 | n/d | |
| Formula VI | 0.5 | 1 | 1 | n/d | |
| Formula VII | 3.5 | 2 | 3 | 15 | nM^{a} |
| Formula VIII | 3.75 | 2 | 3.5 | 15 | nM^{a} |
| Formula IX | 7 | 10 | 14 | 118 | nM^{a} |
| Formula X | 4.35 | 2.5 | 4 | 16 | nM^{a} |
| MTX | 0.35 | 0.85 | 0.85 | 12 | nM^{a} |

^{a}Values for DHFR determined by enzymatic assay.
n/d = not determined

### EXAMPLE 3

### Determination of Cell Proliferation Inhibition IC₅₀ Values

A panel of cancer cell lines was obtained from the DCTP Tumor Repository, National Cancer Institute (Fredrick, Md.) or ATCC (Rockville, Md.). Cell cultures were maintained in Hyclone RPMI 1640 medium (Logan, Utah) supplemented with 10% fetal bovine serum and 20 mM HEPES buffer, pH 7.2, at 37° C., 5% CO2 atmosphere. Cultures were maintained at sub-confluent densities.

Human umbilical vein endothelial cells (HUVEC) were purchased from Clonetics, a division of Cabrex (Walkersville, Md.). Cultures were established from cryopreserved stocks using Clonetics EGM-2 medium supplemented with 20 mM HEPES, pH 7.2, at 37° C., 5% CO₂ atmosphere.

For proliferation assays, cells were seeded with the appropriate medium into 96 well plates at 1,000-2,000 cells per well, depending on cell line, and were incubated overnight. The following day, an amount of test compound, DMSO solution (negative control), or Actinomycin D (positive control) was added to the appropriate wells as 10× concentrated stocks prepared in phosphate buffered saline. The cell plates were then incubated for an additional 2-5 days, dependent on cell line, to allow for proliferation to occur. To measure cell density, 50 μL of WST-1 solution (Roche Applied Science, IN) diluted 1:5 in phosphate buffered saline was added to each well, and the cells incubated for an additional 1-5 hours, again dependent on cell type. Optical density was determined for each well a 450 nm using a Tecan GniosPro plate reader (RTP, NC). The percentage of cell growth was then determined by comparing the cell growth in the presence of test compounds to the DMSO vehicle control treated cells (100% growth) and 10 μL Actinomycin D treated cells (0% growth).

Immediately after the WST-1 determination, the medium was removed from the NCI-H460 and HUVEC cell lines, and the plates stored at −80° C. Using these assay plates, relative DNA amounts in each well were determined using the Cyquant DNA assay kit from R&D Systems (Eugene, Oreg.) following the manufacturer's directions. Results for each compound treatment were compared to DMSO vehicle control (100%) and 10 μL Actinomycin D treated cells (0%).

The data was used to calculate the potency of the test compound, expressed in terms of the fold improvement in cell proliferation IC₅₀ values for the test compound as compared to MTX. These values are set forth in the following table.

TABLE 3
Test Compound Antiproliferative Activity Compared to MTX

| | Fold Improvement | |
|---|---|---|
| | in Cell Proliferation | |
| | IC₅₀ Compared to MTX | |
| | Compound of | c Series |
| Cell Line (tissue type; histology) | Formula III | Derivative |
| SR (lymphoma, pleural effusion) | >4 | N/A |
| OVCAR8 (ovarian; adenocarcinoma) | >2 | N/A |
| SF-268 (central nervous system; | >4 | >1 |
| anaplastic astrocytoma) | | |
| NCI-H460 (lung; large cell carcinoma) | >1 | N/A |
| OVCAR-3 (ovarian; adenocarcinoma) | >3 | >1 |
| Lncap (prostate; carcinoma) | 7 | >2 |
| MCF-7 (mammary; adenocarcinoma) | >1 | N/A |
| HT29 (colon; adenocarcinoma, GRIII) | >6 | >1 |
| K562 (chronic myelogenous carcinoma) | N/A | N/A |
| SkBr (breast cancer) | >2 | N/A |
| NCI/ADR Res (breast; adenocarcinoma) | >1 | N/A |
| SK-Mel 5 (skin; melanoma) | >4 | N/A |
| HUVEC | >2 | N/A |
| BT474 (breast cancer) | >5 | >1 |
| T47D (mammary; carcinoma) | 3 | N/A |
| DMS-114 (N/A; small cell) | >4 | >1 |
| PC-3 (N/A; carcinoma) | >1 | N/A |

Various publications, patent applications and patents are cited herein, the disclosures of which are incorporated by reference in their entireties.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A compound according to the formula 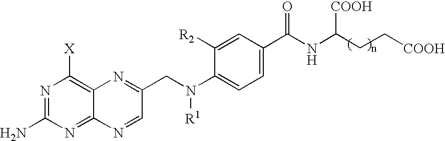
wherein:
X is OH or NH₂;
n=1 or 2;
R₁ is selected from the group consisting of alkenyl, alkynyl, cycloalkylalkyl, heteroarylalkyl, arylalkyl, C₅-C₁₀ alkyl; and
R₂ is selected from the group consisting of H, C₁-C₈ alkyl, CF₃, CF₃O, alkoxy, alkenyl, aralkyl (arylalkyl), heteroarylalkyl, alkynyl, CHO, COCH₃, and halogen;
provided that R₁ is C₅-C₁₀ alkyl when R₂ is H;
or a pharmaceutically acceptable ester, amide, salt, or solvate thereof.

2. A compound according to claim 1, wherein R₁ is 2-propyne.

3. A compound according to claim 1, wherein the compound is selected from the group consisting of:

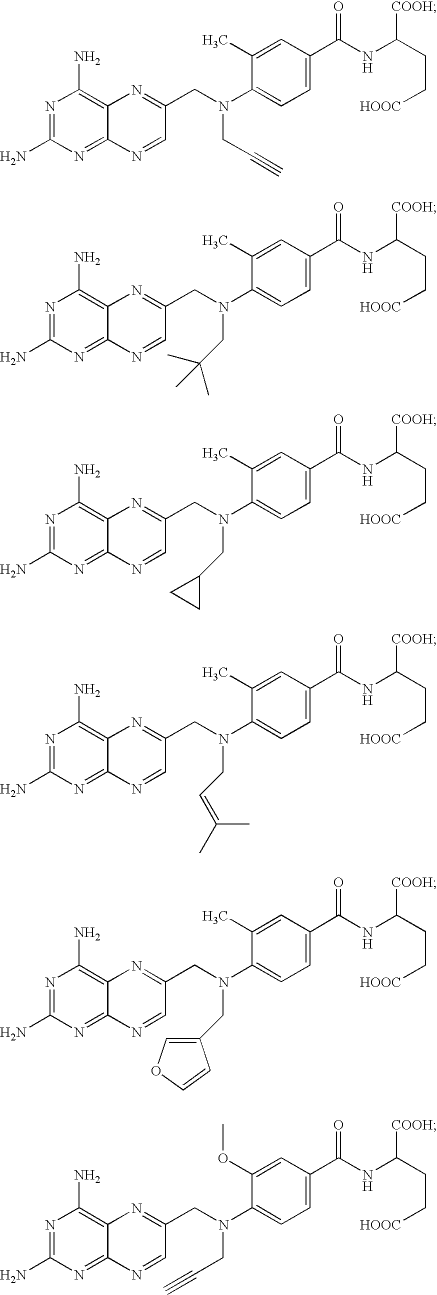

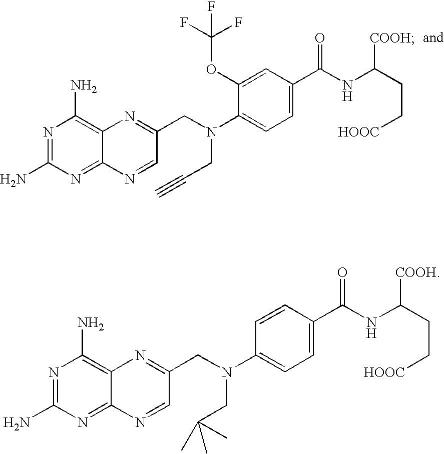

4. A pharmaceutical composition comprising at least one compound of the formula 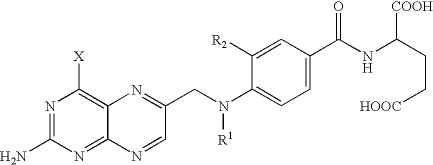
wherein:
X is OH or NH₂;
n=1 or 2;
R₁ is selected from the group consisting of alkenyl, alkynyl, cycloalkylalkyl, heteroarylalkyl, arylalkyl, C₅-C₁₀ alkyl; and
R₂ is selected from the group consisting of H, C₁-C₈ alkyl, CF₃, CF₃O, alkoxy, alkenyl, aralkyl (arylalkyl), heteroarylalkyl, alkynyl, CHO, COCH₃, and halogen;
provided that R₁ is C₅-C₁₀ alkyl when R₂ is H;
or a pharmaceutically acceptable ester, amide, salt, or solvate thereof,
and a pharmaceutically acceptable carrier therefore.

5. A pharmaceutical composition according to claim 4, wherein R₁ is 2-propyne.

6. A pharmaceutical composition according to claim 4, wherein the at least one compound is selected from the group consisting of

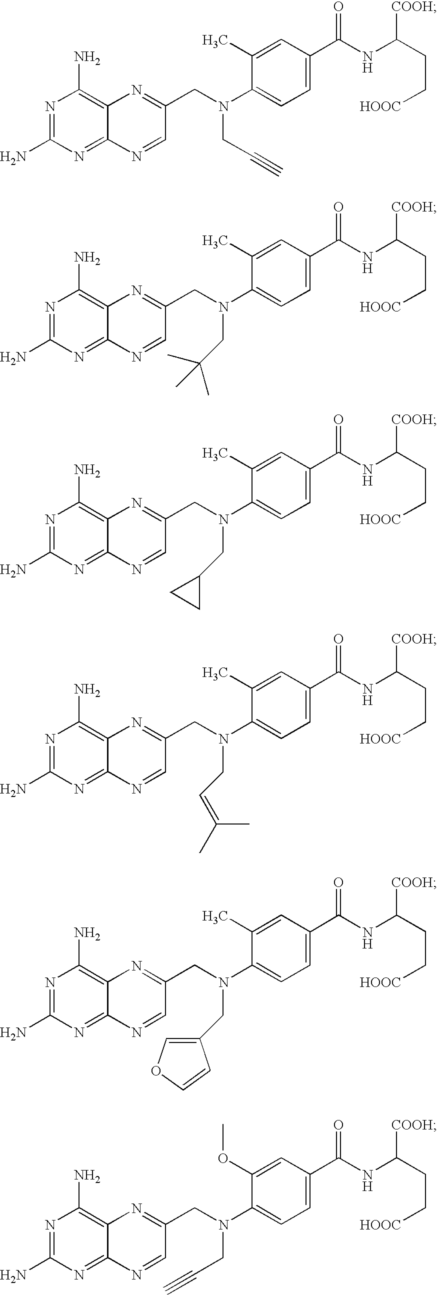

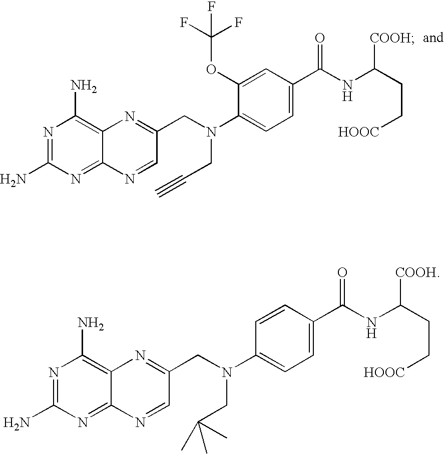

7. A method for inhibiting the activity of at least one protein polypeptide selected from the group consisting of B-Raf, Her2, and EGFR, comprising contacting the polypeptide with an effective amount of at least one compound according to claim 1.

8. The method of claim 7, wherein the inhibition is at least double the inhibition determined for MTX.

9. A method for inhibiting the activity of at least one polypeptide selected from the group consisting of B-Raf, Her2, and EGFR, comprising contacting the polypeptide with an effective amount of the compound according to claim 2.

10. The method of claim 9, wherein the inhibition is at least double the inhibition determined for MTX.

11. A method for inhibiting the activity of at least one polypeptide selected from the group consisting of B-Raf, Her2, and EGFR, comprising contacting the polypeptide with an effective amount of at least one compound according to claim 3.

12. The method of claim 11, wherein the inhibition is at least twice the inhibition determined for MTX.

13. A method for inhibiting proliferation of a cell comprising contacting the cell with an effective amount of at least one compound according to claim 1.

14. The method of claim 13, wherein the inhibition is at least twice the inhibition determined for MTX.

15. A method for inhibiting proliferation of a cell comprising contacting the cell with an effective amount of the compound according to claim 2.

16. The method of claim 15, wherein the inhibition is at least twice the inhibition determined for MTX.

17. A method for inhibiting proliferation of a cell comprising contacting the cell with an effective amount of a compound according to claim 3.

18. The method of claim 17, wherein the inhibition is at least twice that determined for MTX.

19. The method of claim 13, wherein the cell is in vitro.

20. The method of claim 15, wherein the cell is in vitro.

21. The method of claim 17, wherein the cell is in vitro.

22. The method of claim 13, wherein the cell is in vivo.

23. The method of claim 15, wherein the cell is in vivo.

24. The method of claim 17, wherein the cell is in vivo.

25. A compound according to claim 1, wherein the compound has antiproliferative activity.

26. A compound according to claim 2, wherein the compound has antiproliferative activity.

27. A compound according to claim 3, wherein the compound has antiproliferative activity.

28. The compound according to claim 25, wherein said antiproliferative activity is in vitro.

29. The compound according to claim 26, wherein said antiproliferative activity is in vitro.

30. The compound according to claim 27, wherein said antiproliferative activity is in vitro.

31. The compound according to claim 25, wherein said antiproliferative activity is in vivo.

32. The compound of according to claim 26, wherein said antiproliferative activity is in vivo.

33. The compound of according to claim 27, wherein said antiproliferative activity is in vivo.

34. A method of screening for drugs used to improve treatment of a neoplastic, hyperproliferative, or immune disorder, said method comprising:
(a) screening one or more compounds of interest to determine said one or more compounds of interest level of binding to one or more enzymes selected from the group consisting of pyruvate carboxylase and propionyl-CoA carboxylase; and
(b) comparing the level of binding determined in (a) with the level of binding for a compound according to claim 1;
wherein a compound of interest that has a low level of binding to at least one enzyme selected from the group consisting of pyruvate carboxylase and propionyl-CoA carboxylase is identified as a drug that may be used to improve treatment of a neoplastic, hyperproliferative, or immune disorder.

35. A method of screening for drugs used to improve treatment of a neoplastic, hyperproliferative, or immune disorder, said method comprising:
(a) screening one or more compounds of interest to determine said one or more compounds of interest level of binding to one or more enzymes selected from the group consisting of pyruvate carboxylase and propionyl-CoA carboxylase; and
(b) comparing the level of binding determined in (a) with the level of binding for the compound according to claim 2;
wherein a compound of interest that has a low level of binding to at least one enzyme selected from the group consisting of pyruvate carboxylase and propionyl-CoA carboxylase is identified as a drug that may be used to improve treatment of a neoplastic, hyperproliferative, or immune disorder.

36. A method of screening for drugs used to improve treatment of a neoplastic, hyperproliferative, or immune disorder, said method comprising:
(a) screening one or more compounds of interest to determine said one or more compounds of interest level of binding to one or more enzymes selected from the group consisting of pyruvate carboxylase and propionyl-CoA carboxylase; and
(b) comparing the level of binding determined in (a) with the level of binding for a compound according to claim 3;
wherein a compound of interest that has a low level of binding to at least one enzyme selected from the group consisting of pyruvate carboxylase and propionyl-CoA carboxylase is identified as a drug that may be used to improve treatment of a neoplastic, hyperproliferative, or immune disorder.

37. A method of screening for drugs used to improve treatment of a neoplastic, hyperproliferative, or immune disorder, said method comprising:
(a) screening one or more compounds of interest to determine the degree to which said compounds inhibit Her2, EGFR, or B-Raf activity; and
(b) comparing the degree to which said compounds inhibit Her2, EGFR, or B-Raf activity in (a) with the degree to which a compound according to claim 1 inhibits Her2, EGFR, or B-Raf activity;
wherein a compound of interest that inhibits Her2, EGFR, or B-Raf activity to a greater degree than a compound according to claim 1 is identified as a drug that may be used to improve treatment of a neoplastic, hyperproliferative, or immune disorder.

38. A method of screening for drugs used to improve treatment of a neoplastic, hyperproliferative, or immune disorder, said method comprising:
(a) screening one or more compounds of interest to determine the degree to which said compounds inhibit Her2, EGFR, or B-Raf activity; and
(b) comparing the degree to which said compounds inhibit Her2, EGFR, or B-Raf activity in (a) with the degree to which the compound according to claim 2 inhibits Her2, EGFR, or B-Raf activity;
wherein a compound of interest that inhibits Her2, EGFR, or B-Raf activity to a greater degree than the compound according to claim 2 is identified as a drug that may be used to improve treatment of a neoplastic, hyperproliferative, or immune disorder.

39. A method of screening for drugs used to improve treatment of a neoplastic, hyperproliferative, or immune disorder, said method comprising:
(a) screening one or more compounds of interest to determine the degree to which said compounds inhibit Her2, EGFR, or B-Raf activity; and
(b) comparing the degree to which said compounds inhibit Her2, EGFR, or B-Raf activity in (a) with the degree to which a compound according to claim 3 inhibits Her2, EGFR, or B-Raf activity;
wherein a compound of interest that inhibits Her2, EGFR, or B-Raf activity to a greater degree than a compound according to claim 3 is identified as a drug that may be used to improve treatment of a neoplastic, hyperproliferative, or immune disorder.

40. A method of screening for drugs used to improve treatment of a neoplastic, hyperproliferative, or immune disorder, said method comprising:
(a) screening one or more compounds of interest to determine said compounds antiproliferative activity; and
(b) comparing the antiproliferative activity in (a) with the antiproliferative activity of a compound according to claim 1;
wherein a compound of interest that exhibits a greater antiproliferative activity than a compound according to claim 1 is identified as a drug that may be used to improve treatment of a neoplastic, hyperproliferative, or immune disorder.

41. A method of screening for drugs used to improve treatment of a neoplastic, hyperproliferative, or immune disorder, said method comprising:
(a) screening one or more compounds of interest to determine said compounds antiproliferative activity; and
(b) comparing the antiproliferative activity in (a) with the antiproliferative activity of the compound according to claim 2;
wherein a compound of interest that exhibits a greater antiproliferative activity than the compound according to claim 2 is identified as a drug that may be used to improve treatment of a neoplastic, hyperproliferative, or immune disorder.

42. A method of screening for drugs used to improve treatment of a neoplastic, hyperproliferative, or immune disorder, said method comprising:
(a) screening one or more compounds of interest to determine said compounds antiproliferative activity; and
(b) comparing the antiproliferative activity in (a) with the antiproliferative activity of a compound according to claim 3;
wherein a compound of interest that exhibits a greater antiproliferative activity than a compound according to claim 3 is identified as a drug that may be used to improve treatment of a neoplastic, hyperproliferative, or immune disorder.
